(19) United States
(12) Patent Application Publication
(10) Pub. No.: US 2007/0037886 A1
(43) Pub. Date: Feb. 15, 2007

(54) Bone marrow erythroid progenitor cell(s) differentiation inducer

(75) Inventors: Sen Takeshita, Kawasaki-shi (JP); Yuzuru Eto, Kawasaki-shi (JP); Yoshiyuki Takahara, Kawasaki-shi (JP); Kiyonobu Kamiya, Kawasaki-shi (JP); Kazumi Tashiro, Kawasaki-shi (JP); Maki Sugiyama, Kawasaki-shi (JP); Hiroshi Yamamoto, Kawasaki-shi (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/411,024

(22) Filed: Apr. 26, 2006

Related U.S. Application Data
(60) Provisional application No. 60/678,178, filed on May 6, 2005.

(30) Foreign Application Priority Data
Apr. 26, 2005 (JP) 2005-128204

(51) Int. Cl. A61K 31/198

(52) U.S. Cl. 514/565

(57) ABSTRACT

Bone marrow erythroid progenitor cell(s) differentiation may be induced and anemia may be treated by administering arginine. Arginine is an excellent differentiation inducer, because it has high safety, can be orally administered, and is widely used.

### CROSS REFERENCES TO RELATED APPLICATIONS

This application claims priority to U.S. Provisional Patent Application No. 60/678,178, filed on May 6, 2005, and Japanese Patent Application No. 2005-128204, filed on Apr. 26, 2005, both of which are incorporated herein by reference in their entireties.

### BACKGROUND OF THE INVENTION

1. Field of the Invention

The present invention relates to bone marrow erythroid progenitor cell(s) differentiation inducers and therapeutic agents for anemia which contain the same. The present invention also relates to methods for treating and/or preventing anemia.

2. Discussion of the Background

Erythropoiesis is an essential process required to maintain homeostasis of the number of red blood cells. Red blood cells have an average life span of about 120 days in humans, and worn out red blood cells are continuously removed from the circulation system. Therefore, about 100 billion red blood cells are newly produced daily in adults. There have been many investigations of the generation of red blood cells, which are described in a number of books. As a typical example, a summary is excerpted from “Hematology, Chugai Igakusha”, which will be shown below.

There are multipotent stem cells capable of differentiating into many different types of blood cell systems in the bone marrow, and a portion of the multipotent stem cells differentiate into erythroid progenitors determined to differentiate into the erythroid system. The most primitive identifiable erythroid progenitors are the burst forming unit-erythroid (BFU-E) and the colony forming unit-erythroid (CFU-E), which is a more differentiated cell. Beyond the CFU-E stage, the cells differentiate into proerythroblasts, basophilic erythroblasts, polychromatic erythroblasts, and orthochromatic erythroblasts while dividing, and reticulocytes differentiate into mature red blood cells by enucleation.

At the BFU-E and CFU-E stages, the direction of differentiation into the erythroid system is completely determined, therefore the cells never differentiate into a blood cell system other than the erythroid system. Thus, as the number of BFU-E and CFU-E increases, erythropoiesis is promoted.

As for erythropoiesis, homeostasis is principally regulated by erythropoietin (EPO), which is a hematopoietic factor. EPO is mainly produced in the kidney and circulates in the blood and acts on CFU-E in the bone marrow and stimulates proliferation and differentiation of CFU-E, thereby promoting erythropoiesis. When EPO is not produced at a normal level and running short, the amount of CFU-E decreases and erythropoiesis is reduced thereby causing anemia. Anemia is the pathological consequence of insufficient hemoglobin levels to meet the oxygen transport requirements of the body and causes clinical symptoms such as less incentive to work, fatigability, shortness of breath, lightheadedness and palpitation. Therefore there is a demand for improving such symptoms. It is known that various diseases cause anemia due to insufficient EPO, and the most classical example is a kidney disease. In patients with chronic renal failure, EPO production is reduced due to renal damage, whereby the patients exhibit anemia. Many of the patients with chronic renal failure are dialysis patients who require frequent dialysis for renal function replacement, and 90% of the dialysis patients have anemia. At present, as a method of treating anemia, administration of recombinant human EPO (rHuEPO) is widely used, and 90% of the dialysis patients are administered rHuEPO. In many of the patients, an effect on improving anemia has been confirmed.

The effect of rHuEPO on improving anemia is high, however, several problems have arisen accompanying the expansion of the clinical use thereof. One is the cost for long-term treatment thereof. The typical dose of rHuEPO is 9000 IU/week at most, which costs about 12,000 yen on an NHI drug price basis. However, unless the primary disease causing anemia is treated, it is administered over a long period of time. Therefore, the burden on the patients and health care system is great in terms of the cost. Further, because EPO has to be intravenously administered, the patients need to go to the hospital every time they receive the administration, etc., which is also a cause of why the burden on patients is great. Further, it is known that there are about 10 to 20% of patients whose EPO level in the blood is increased by the administration of rHuEPO, but whose reactivity against EPO is low, therefore who need a high dose of rHuEPO for alleviating anemia or whose anemia is hardly improved at all (these patients are called EPO-unresponsive patients). It is considered that for alleviating anemia in the EPO-unresponsive patients, a bone marrow erythroid progenitor cell(s) differentiation inducer, which increases CFU-E based on a mechanism of action which is different from that of EPO, would be effective. Further, by the concomitant use of the bone marrow erythroid progenitor cell(s) differentiation inducer, an effect of reducing the required dose of rHuEPO is also expected.

Moreover, for an individual who collects and stores his/her own blood for a future use (an individual who stores his/her own blood), the bone marrow erythroid progenitor cell(s) differentiation inducer can be used as a means of promoting hematopoietic action promptly after the collection of blood.

In Int. J. Toxicol., vol. 23, pp. 101-105 (2004), it is described that the amount of hemoglobin and red blood cells was increased by repeatedly administering arginine to normal rats. However, it is not known whether it is caused by an effect of enhancing erythroid regeneration (erythropoiesis), or an effect of prolonging the life span of red blood cells, that is, the mechanism of action is not known. Therefore, the clinical effectiveness, for example, the type of anemia in which the inducer is effective, is not clear. In Igakuno Ayumi, 211, No. 8 (2004), it is described that the condition of anemia was improved (the number of red blood cells came close to a normal level) by administering arginine to patients with renal anemia, and that the mechanism of action thereof resides in the promotion of renal EPO production by arginine. Accordingly, it can be inferred naturally and easily that the phenomenon of increasing the number of red blood cells observed in normal rats described in Int. J. Toxicol., vol. 23, pp. 101-105 (2004) is caused by the promotion of renal EPO production in the rats by arginine. That is, for researchers in this field, it is difficult to predict that arginine has a hematopoietic action by directly acting on the bone marrow to induce proliferation of erythroid progenitors. Further, it is a natural consequence that the researchers would think that the type of anemia in which arginine is effective is limited to one in which the promotion of EPO production is involved in the therapeutic effect. In this way, the researchers would have never thought that arginine has a myelopoietic action, that arginine can also be applied to a treatment of anemia for EPO-unresponsive patients, or the dose of EPO could be reduced by a synergistic effect with EPO and the like.

### SUMMARY OF THE INVENTION

Accordingly, it is one object of the present invention to provide novel bone marrow erythroid progenitor cell(s) differentiation inducers.

It is another object of the present invention to provide novel therapeutic agents for anemia.

It is another object of the present invention to provide novel methods of treating and/or preventing anemia.

These and other objects, which will become apparent during the following detailed description, have been achieved by the inventors' discovery that arginine, which is an amino acid, has an action of increasing CFU-E.

More specifically, for measuring CFU-E, the in vitro colony assay method using a methylcellulose semi-solid medium is generally used, and it is the most suitable experimental system for discovering an action of increasing CFU-E. The present inventors employed this method and measured the number of CFU-E colonies when 600 μM arginine was added to isolated mouse bone marrow cells, and as a result, they found that the number of CFU-E significantly increases compared with the case of an average concentration of arginine in the blood, 170 μM. In the case of oral administration to rats, the blood arginine concentration reaches about 600 μM by a single-dose administration of arginine at 1.2 g/kg. Therefore, it is expected that the number of CFU-E increases in a dose-dependent manner by such an oral administration. Also in humans, it is expected that the number of CFU-E increases as the concentration of arginine in the blood increases by the oral administration.

Accordingly, the present invention provides the following:

(1) A bone marrow erythroid progenitor cell(s) differentiation inducer, comprising arginine as an active ingredient.

(2) A therapeutic agent for anemia, comprising the differentiation inducer according to (1).

(3) The therapeutic agent for anemia according to (2), which is for hemodialysis or peritoneal dialysis.

(4) The therapeutic agent for anemia according to (2), which is for oral administration.

(5) The therapeutic agent for anemia according to (2), which is for enteral administration.

(6) The therapeutic agent for anemia according to any one of (2) to (5), which is for treating renal anemia.

(7) The therapeutic agent for anemia according to any one of (2) to (5), which is for erythropoietin-unresponsive patients.

(8) The therapeutic agent for anemia according to any one of (2) to (7), further comprising an erythropoietin-like substance in combination.

(9) The therapeutic agent for anemia according to (8), wherein the erythropoietin-like substance is erythropoietin.

(10) The therapeutic agent for anemia according to (8), wherein the erythropoietin-like substance is an erythropoietin mimetic peptide.

(11) The therapeutic agent for anemia according to (8), wherein the erythropoietin-like substance is an erythropoietin production promoter.

(12) The therapeutic agent for anemia according to any one of (8) to (11), which is a combination preparation.

(13) The therapeutic agent for anemia according to (8), which is a kit composed of a medicament comprising arginine and a medicament comprising an erythropoietin-like substance.

(14) The therapeutic agent for anemia according to (13), wherein the erythropoietin-like substance is erythropoietin.

(15) The therapeutic agent for anemia according to (13), wherein the erythropoietin-like substance is an erythropoietin mimetic peptide.

(16) The therapeutic agent for anemia according to (13), wherein the erythropoietin-like substance is an erythropoietin production promoter.

(17) An agent for enhancing the effect of an erythropoietin-like substance, comprising arginine as an active ingredient.

(18) The effect-enhancing agent according to (17), wherein the erythropoietin-like substance is erythropoietin.

(19) The effect-enhancing agent according to (17), wherein the erythropoietin-like substance is an erythropoietin mimetic peptide.

(20) The effect-enhancing agent according to (17), wherein the erythropoietin-like substance is an erythropoietin production promoter.

(21) An agent for reducing the dose of an erythropoietin-like substance, comprising arginine as an active ingredient.

(22) The dose-reducing agent according to (21), wherein the erythropoietin-like substance is erythropoietin.

(23) The dose-reducing agent according to (21), wherein the erythropoietin-like substance is an erythropoietin mimetic peptide.

(24) The dose-reducing agent according to (21), wherein the erythropoietin-like substance is an erythropoietin production promoter.

(25) A hematopoiesis promoter for an individual who stores his/her own blood, comprising arginine as an active ingredient.

(26) A method for inducing bone marrow erythroid progenitor cells differentiation inducer, comprising administering an effective amount of arginine to a subject in need thereof.

(27) A method of treating and/or preventing anemia, comprising administering an effective amount of arginine to a subject in need thereof.

(28) The method according to (27), wherein said arginine is administered in an amount of 0.1 to 12 g per day.

(29) The method according to (27), wherein said arginine is administered in an amount of 0.5 to 6 g per day.

(30) The method according to (27), wherein said subject undergoes hemodialysis or peritoneal dialysis.

(31) The method according to (27), wherein said arginine is administered orally.

(32) The method according to (27), wherein said arginine is administered enterally.

(33) The method according to any one of (26) to (32), wherein said subject suffers from renal anemia.

(34) The method according to any one of (26) to (33), wherein said subject is erythropoietin-unresponsive.

(35) The method according to (34), wherein said subject exhibits an increase in Hct value of less than 3% after 4 to 8 weeks of administration of 9000 IU/week of EPO.

(36) The method according to any one of (26) to (35), further comprising administering an erythropoietin-like substance.

(37) The method according to (36), wherein said erythropoietin-like substance is administered in an amount of 1500 to 9000 IU/week.

(38) The method according to (36), wherein the erythropoietin-like substance is erythropoietin.

(39) The method according to (36), wherein the erythropoietin-like substance is an erythropoietin mimetic peptide.

(40) The method according to (36), wherein the erythropoietin-like substance is an erythropoietin production promoter.

(41) The method according to any one of (36) to (40), wherein said arginine and said erythropoietin-like substance are administered separately.

(42) The method according to (36), wherein said arginine and said erythropoietin-like substance are administered in a combined composition.

(43) The method according to (42), wherein the erythropoietin-like substance is erythropoietin.

(44) The method according to (42), wherein the erythropoietin-like substance is an erythropoietin mimetic peptide.

(45) The method according to (42), wherein the erythropoietin-like substance is an erythropoietin production promoter.

(46) A method for enhancing the effect of an erythropoietin-like substance in treating and/or preventing anemia, comprising administering an effective amount of arginine to a subject in need thereof.

(47) The method according to (46), wherein said arginine is administered in an amount of 0.1 to 12 g per day.

(48) The method according to (46), wherein said arginine is administered in an amount of 0.5 to 6 g per day.

(49) The method according to (46), wherein the erythropoietin-like substance is erythropoietin.

(50) The method according to (46), wherein the erythropoietin-like substance is an erythropoietin mimetic peptide.

(51) The method according to (46), wherein the erythropoietin-like substance is an erythropoietin production promoter.

(52) A method for reducing the dose of an erythropoietin-like substance effective for treating and/or preventing anemia, comprising administering an effective amount of arginine to a subject in need thereof.

(53) The method according to (52), wherein said arginine is administered in an amount of 0.1 to 12 g per day.

(54) The method according to (52), wherein said arginine is administered in an amount of 0.5 to 6 g per day.

(55) The method according to (52), wherein the erythropoietin-like substance is erythropoietin.

(56) The method according to (52), wherein the erythropoietin-like substance is an erythropoietin mimetic peptide.

(57) The method according to (52), wherein the erythropoietin-like substance is an erythropoietin production promoter.

(58) A method of promoting hematopoiesis for an individual who stores his/her own blood, comprising administering an effective amount of arginine to said individual.

(59) The method according to (58), wherein said arginine is administered in an amount of 0.1 to 12 g per day.

(60) The method according to (58), wherein said arginine is administered in an amount of 0.5 to 6 g per day.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIGS. 1A and 1B show the CFU-E-colony stimulating activity of arginine (EPO). As shown in FIG. 1A, arginine increased the number of CFU-E under the condition in which the amount of EPO (1 U/ml) is sufficient. As shown in FIG. 1B, the amount of EPO for the highest action is 0.5 U/ml or higher, and it is apparent that 1 U/ml of EPO is a sufficient amount.

FIGS. 2A and 2B show the CFU-E-colony stimulating activity of arginine (EPO mimetic peptide). As shown in FIG. 2A, arginine increased the number of CFU-E under the condition in which the amount of EMP1 (EPO mimetic peptide, 30 μM) is sufficient. As shown in FIG. 2B, the amount of EMP1 for the highest action is 10 μM or higher, and it is apparent that 30 μM of EMP1 is a sufficient amount.

### DETAILED DESCRIPTION OF THE PREFERRED EMBODIMENTS

The bone marrow erythroid progenitor cell(s) differentiation inducer according to the present invention comprises arginine as an active ingredient. The arginine may be arginine and/or a physiologically acceptable salt thereof. Examples of suitable salts include acid addition salts, such as a hydrochloride, a hydrobromide, a hydroiodide; acid addition salts of arginine with citric acid, sulfiric acid, phosphoric acid, methanesulfonic acid, bezenesulfonic acid, toluenesulfonic acid; acid addition salts of arginine with an acidic amino acid such as glutamatic acid and aspartic acid; and the like. Other suitable salts include a sodium salt, a potassium salt, an ammonium salt, a mono-, di-, or trialkylammonium salt, a mono-, di, or tri(hydroxyalkyl)ammonium salt, etc. However, it is not limited to these. As for an isomer of the active ingredient, any of the L-form, D-form and DL-form can be used. However, the L-form is preferred because it is naturally occurring. For example, as the arginine, the free-form of L-arginine and L-arginine monohydrochloride may be included in combination.

The bone marrow erythroid progenitor cell(s) progenitor differentiation inducer of the present invention can be prepared in any of known forms as well as various forms of pharmaceutical preparations to be discovered in the future for, for example, oral administration, intraperitoneal administration, percutaneous administration, subcutaneous administration, intravenous administration, inhalation, and the like. In the myeloerythroid progenitor differentiation inducer of the present invention, arginine can be used alone. However, as a pharmaceutical preparation comprising arginine as an active ingredient, it can be prepared in any of various forms by suitably employing known methods and methods developed in the future.

A method of administering the myeloerythroid progenitor differentiation inducer of the present invention is not particularly limited, however, oral administration is preferred. In this case, the dose varies depending on the hemoglobin level, which becomes an indicator of anemia for a patient to be administered, the age and the like, however, in the case of adults, it is about 0.1 to 12 g, more preferably about 0.5 to 6 g per day.

The bone marrow erythroid progenitor cell(s) differentiation inducer of the present invention can be used as an active ingredient of a pharmaceutical product to be used for treating or preventing a variety of diseases caused by a decrease in erythropoiesis or as a constituent of a food or a medical food. Examples of the disease in which the bone marrow erythroid progenitor cell(s) differentiation inducer of the present invention is expected to be effective include renal anemia, iron-deficiency anemia, hemolytic anemia, aplastic anemia, pernicious anemia, bleeding anemia, and anemia accompanying a treatment with an anti-cancer agent. Among these diseases, the myeloerythroid progenitor differentiation inducer of the present invention is useful for a treatment of renal anemia, particularly EPO-unresponsive renal anemia.

The term “EPO unresponsiveness” as used herein means a state in which a sufficient effect is not expressed even if EPO is administered in an amount at which an effect is expected, and it is not particularly limited to the dose of EPO. Preferably, the term “EPO unresponsiveness” means a state in which a sufficient effect is not expressed even if EPO is administered at 9000 IU/week or more. “A state in which a sufficient effect is not expressed” means preferably the case where an increase in the Hct value after 4 to 8-weeks of administration is less than 3%. In general, the definition of an EPO-unresponsive patient varies depending on the race, the lifestyle habit or the like, however, the present invention is not limited to the definition. For example, in the U.S., a patient whose hemoglobin (“Hb”) level does not reach 10 to 12 g/dl even if EPO has been administered at 300 to 450 IU/kg/week for 4 to 6 months is defined as an EPO-unresponsive patient in the guideline (see, Am J. Kidney Dis., 30 Suppl. 3, (1997)). However, in a general dosage regimen, a patient whose Hb level is not increased by 2 g/dl and whose Hb level does not reach 10 to 12 g/dl even if EPO has been administered at 150 to 300 IU/kg/week for 2 months is defined as an EPO-unresponsive patient (the package insert of EPOGEN, a tradename for rHuEPO). In the Europe, a patient whose hematocrit value (“Hct,” the percentage of the volume of a blood sample occupied by cells) does not reach a desired value even if EPO has been subcutaneously administered at 300 IU/kg/week or more is defined as an EPO-unresponsive patient in the guideline (see, Nephrol. Dial. Transplant, vol. 14 Suppl. 5, pp. 25-27 (1999)). In Japan, the required dose of EPO is 9000 IU/week or more, and the case where an increase in the Hct value after 4 to 8-weeks of administration is less than 3% is considered as EPO unresponsiveness. Also in countries other than the above-mentioned countries, the case where an effect on improving anemia cannot be expected even at the recommended maximum dosage regimen is defined as an EPO-unresponsive patient. For such an EPO-unresponsive patient, administration of the bone marrow erythroid progenitor cell(s) differentiation inducer of the present invention is essential.

Among patients administered with EPO other than EPO-unresponsive patients, for patients for whom the required dose of EPO is the maximum dosage regimen, preferably 1500 to 9000 IU/week, enhancement of improvement of anemia by the myeloerythroid progenitor differentiation inducer of the present invention is expected. Therefore, it is expected that the required dose of EPO at a treatment for improving anemia by EPO alone can be reduced. It is expected that the enhancement of EPO and the reduction of the dose of EPO by administering the myeloerythroid progenitor differentiation inducer of the present invention is effective even in the case where sufficient improvement of anemia is not observed for a standard administration period (an increase in the Hct value is less than about 3% after 4 to 8 weeks) at a standard dose of EPO at the initiation of EPO administration. Further, it is expected that the bone marrow erythroid progenitor cell(s) differentiation inducer of the present invention is effective also in the case where sufficient improvement of anemia is not observed for a standard administration period even when the dose of EPO thereafter is increased to the maximum dose to be generally administered. As a method of administration for enhancement of EPO and reduction of the dose of EPO described above, oral administration and parenteral administration can be employed. By administering the bone marrow erythroid progenitor cell(s) differentiation inducer of the present invention, improvement of anemia is expected for patients who undergo or do not undergo dialysis among patients with renal anemia, and as a method of administration, oral administration and parenteral administration can be employed.

The “erythropoietin-like substance” refers to a substance that exerts or induces erythropoietin-like action in vivo when it is administered or taken in vivo or the like.

Examples thereof include erythropoietin, an erythropoietin mimetic peptide, an erythropoietin production promoter and the like, however, it is not particularly limited to these.

The erythropoietin may be any of a natural type, a genetically engineered type, and a modified type. For example, as the genetically engineered type, epoetin alfa (genetical recombination) or epoetin beta (genetical recombination), each of which is a glycoprotein (molecular weight: about 30,000) comprising 165 amino acid residues (C₈₀₉H₁₃₀₁N₂₂₉O₂₄₀S₅, molecular weight: 18,235.96) produced in a chinese hamster ovarian cell by expressing human erythropoietin cDNA derived from human hepatocyte mRNA, or the like may be employed.

The term “erythropoietin mimetic peptide” refers to a peptide that binds to and activates an EPO receptor, or behaves as an EPO agonist. For example, it includes a peptide of 10 to 40 amino acid residues in length, which comprises an amino acid sequence, X₃X₄X₅GPX₆TWX₇X₈, disclosed in WO 96/40749, which is incorporated herein by reference in its entirety. Here, each amino acid is indicated by standard one letter abbreviation; X₃ is C; X₄ is R, H, L, or W; X₅ is M, F, or I; X₆ is independently selected from any one of the 20 genetically encoded L-amino acids; X₇ is D, E, I, L, or V; and X₈ is C.

More specifically, the EPO mimetic peptide refers to a peptide having a peptide that satisfies the following requirements as a component:

1. A peptide of 10 to 40 amino acid residues in length, which binds to an EPO receptor and comprises an amino acid sequence, X₃X₄X₅GPX₆TWX₇X₈, wherein each amino acid is indicated by standard one letter abbreviation; X₆ is independently selected from any one of the 20 genetically encoded L-amino acids; X₃ is C; X₄ is R, H, L, or W; X₅ is M, F, or I; X₇ is D, E, I, L, or V; and X₈ is C.

2. The peptide according to 1, comprising an amino acid sequence, YX₂X₃X₄X₅GPX₆TWX₇X₈, wherein each amino acid is indicated by standard one letter abbreviation; each of X₂ and X₆ is independently selected from any one of the 20 genetically encoded L-amino acids; X₃ is C; X₄ is R, H, L, or W; X₅ is M, F, or I; X₇ is D, E, I, L, or V; and X₈ is C.

3. The peptide according to 2, comprising an amino acid sequence, X₁YX₂X₃X₄X₅GPX₆TWX₇X₈X₉X₁₀X₁₁, wherein each amino acid is indicated by standard one letter abbreviation; each of X₁, X₂, X₆, X₉, X₁₀ and X₁₁, is independently selected from any one of the 20 genetically encoded L-amino acids; X₃ is C; X₄ is R, H, L, or W; X₅ is M, F, or I; X₇ is D, E, I, L, or V; and X₈ is C.

4. The peptide according to 3, wherein X₄ is R or H; X₅ is F or M; X₆ is I, L, T, M, E or V; X₇is D or V; X₉ is G, K, L, Q, R, S or T; and X₁₀ is A, G, P, R or Y.

5. The peptide according to 4, wherein X₁ is D, E, L, N, S, T or V; X₂ is A, H, K, L, M, S or T; X₄ is R or H; X₉ is K, R, S or T; and X₁₀ is P.

6. The peptide according to 1 which is selected from the group consisting of the following peptides:

GGLYLCRFGPVTWDCGYKGG;
GGTYSCHFGPLTWVCKPQGG;
GGTYSCHFGPLTWVCKPQ;
GGDYHCRMGPLTWVCKPLGG;
VGNYMCHFGPITWVCRPGGG;
GGNYMCHFGPITWVCRPGGG;
GGVYACRMGPITWVCSPLGG;
VGNYMAHMGPITWVCRPGG;
GGPHHVYACRMGPLTWIC;
GGTYSCHFGPLTWVCKPQ;
GGLYACHMGPMTWVCQPLRG;
TIAQYICYMGPETWECRPSPKA;
YSCHFGPLTWVCK;
YCHFGPLTWVC;
Ac-GGTYSCHFGPLTWVCKPQGG;
GGCRIGPITWVCGG;
LGRKYSCHFGPLTWVCQPAKKD;
GGTASCHFGPLTWVCKPQGG;
GGNYYCRFGPITFECHPTGG;
GGEYLCRMGPMTWVCTPVGG;
GGLYTCRMGPITWVCLPAGG;
GGTTSCHFGPLTWVCKPQGG;
GGTFSCHFGPLTWVCKPQGG;
GGTYSCHFGALTWVCKPQGG;
GGTYSCHFGPLAWVCKPQGG;
GGTYSCHFAPLTWVCKPQGG;
GGTYSCHFGPATWVCKPQGG;
GGTYSCHFGPLTAVCKPQGG;
GGTYSCHFGPLTFVCKPQGG;
TYSCHFGPLTWVCKPQ;
YSCHFGPLTWVCKP;
SCHFGPLTWVCK;
GGTYSCFGPLTWVCKPQGG;
TYSCHFGPLTWVCKPQGG;
YSCHFGPLTWVC;
GGTYSCHFGPLTFVCKPQGG;
and
HFGPLTWV.

7. The peptide according to 1 which is selected from the group consisting of the following peptides:

GGLYLCRFGPVTWDCGYKGG;
GGTYSCHFGPLTWVCKPQGG;
GGDYHCRMGPLTWVCKPLGG;
VGNYMCHFGPITWVCRPGGG;
GGVYACRMGPITWVCSPLGG;
VGNYMAHMGPITWVCRPGG;
GGPHHVYACRMGPLTWIC;
GGTYSCHFGPLTWVCKPQ;
GGLYACHMGPMTWVCQPLRG;
TIAQYICYMGPETWECRPSPKA;
YSCHFGPLTWVCK;
YCHFGPLTWVC;
and
HFGPLTWV.

8. The peptide according to 1, wherein the amino acid sequence is cyclized.

9. The peptide according to 8 which is selected from the group consisting of the following peptides:

GGTYSCHFGPLTWVCKPQGG;
GGTYSCHFGPLTWVCKPQ;
GGLYACHMGPMTWVCQPLRG;
TIAQYICYMGPETWECRPSPKA;
YSCHFGPLTWVCK;
YCHFGPLTWVC;
Ac-GGTYSCHFGPLTWVCKPQGG;
GGCRIGPITWVCGG;
LGRKYSCHFGPLTWVCQPAKKD;
GGTASCHFGPLTWVCKPQGG;
GGNYYCRFGPITFECHPTGG;
GGEYLCRMGPMTWVCTPVGG;
GGLYTCRMGPITWVCLPAGG;
GGTTSCHFGPLTWVCKPQGG;
GGTFSCHFGPLTWVCKPQGG;
GGTYSCHFGALTWVCKPQGG;
GGTYSCHFGPLAWVCKPQGG;
GGTYSCHFAPLTWVCKPQGG;
GGTYSCHFGPATWVCKPQGG;
GGTYSCHFGPLTAVCKPQGG;
GGTYSCHFGPLTFVCKPQGG;
TYSCHFGPLTWVCKPQ;
YSCHFGPLTWVCKP;
YSCHFGALTWVCK;
SCHFGPLTWVCK;
GGTYSEHFGPLTWVKKPQGG;
GGTYSCFGPLTWVCKPQGG;
TYSCHFGPLTWVCKPQGG;
YSCHFGPLTWVC;
GGTYSCHFGPLTFVCKPQGG; and

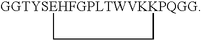

10. The peptide according to 1, wherein the amino acid sequence is dimerized.

11. The peptide according to 10, wherein the peptide comprises the following amino acid sequence:

GGTYSCHFGPLTWVCKPQGG
| |
GGTYSCHFGPLTWVCKPQGG.

Suitable erythropoietin mimetic peptides also include those disclosed in U.S. Pat. No. 5,830,851 (which is incorporated herein by reference in its entirety), i.e., peptides of 10 to 40 or more amino acid residues in length and having the sequence X₃X₄X₅GPX₆TWX₇X₈ (SEQ ID NO:252, in U.S. Pat. No. 5,830,851) where each amino acid is indicated by standard one letter abbreviation; X₃ is C; X₄ is R, H, L, or W; X₅ is M, F, or I; X₆ is independently selected from any one of the 20 genetically coded L-amino acids; X₇ is D, E, I, L, or V; and X₈ is C, which bind and activate the erythropoietin receptor (EPO-R) or otherwise act as an EPO agonist.

More specifically, the EPO mimetic peptide includes peptides having a peptide that satisfies the following requirements as a component:

1. A peptide of 10 to 40 amino acid residues in length that binds to erythropoietin receptor and comprises a sequence of anmio acids X₃X₄X₅GPX₆TWX₇X₈ (SEQ ID NO: 1, in U.S. Pat. No. 5,830,851) where each amino acid is indicated by standard one letter abbreviation; X₆ is independently selected from any one of the 20 genetically coded L-amino acids; X₃ is C; X₄ is R, H, L, or W; X₅ is M, F, I; X₇ is D, E, I, L, or V; and X₈ is C.

2. The peptide according to 1 which comprises a sequence of amino acids YX₂X₃X₄X₅GPX₆TWX₇X₈ (SEQ ID NO:253, in U.S. Pat. No. 5,830,851) where each amino acid is indicated by standard one letter abbreviation; each X₂ and X₆ is independently selected from any one of the 20 genetically coded L-amino acids; X₃ is C; X₄ is R, H, L, or W; X₅ is M, F, or I; X₇ is D, E, I, L, or V; and X₈ is C.

3. The peptide according to 1 which comprises a sequence of amino acids XIYX₂X₃X₄X₅GPX₆TWX₇X₈X₉X₁₀X₁₁ (SEQ ID NO:254, in U.S. Pat. No. 5,830,851) where each amino acid is indicated by standard one letter abbreviation; each X₁, X₂, X₆, X₉, X₁₀, and X₁₁, is independently selected from any one of the 20 genetically coded L-amino acids; X₃ is C; X₄ is R, H, L, or W; X₅ is M, F, or I; X₇ is D, E, I, L, or V; and X₈ is C.

4. The peptide according to 3, wherein X₄ is R or H; X₅ is F or M; X₆ is I, L, T, M, or V; X₇ is D or V; X₉ is G, K, L, Q, R, S, or T; and X₁₀ is A, G, P, R, or Y.

5. The peptide according to 4, wherein X₁ is D, E, L, N, S, T, or V; X₂ is A, H, K, L, M, S, or T; X₄ is R or H; X₉ is K, R, S, or T; and X₁₀ is P.

6. The peptide according to 1, wherein the peptide is

GGTYSCHFGPLTWVCKPQGG (SEQ ID NO:8, in U.S. Pat.
No. 5,830,851).

Suitable erythropoietin mimetic peptides also include those disclosed in U.S. Pat. No. 5,986,047 (which is incorporated herein by reference in its entirety), i.e., peptides of 10 to 40 or more amino acid residues in length and having the sequence X₃X₄X₅GPX₆TWX₇X₈ (SEQ ID NO:252, in U.S. Pat. No. 5,986,047) where each amino acid is indicated by standard one letter abbreviation; X₃ is C; X₄ is R, H, L, or W; X₅ is M, F, or I; X₆ is independently selected from any one of the 20 genetically coded L-amino acids; X₇ is D, E, I, L, or V; and X₈ is C, which bind and activate the erythropoietin receptor (EPO-R) or otherwise act as an EPO agonist.

More specifically, the EPO mimetic peptide includes peptides having a peptide that satisfies the following requirements as a component:

1. A peptide dimer comprising two monomeric peptides of 10 to about 40 amino acids in length that bind to erythropoietin receptor, each monomeric peptide comprising a sequence of amino acids

X₃X₄X₅GPX₆TWX₇X₈ (SEQ ID NO:252, in U.S. Pat. No.
5,986,047)

where each amino acid is indicated by standard one letter abbreviation; X₆ is independently selected from any one of the 20 genetically coded L-amino acids; X₃ is C; X₄ is R, H, L, or W; X₅ is M, F, or I; X₇ is D, E, I, L, or V; and X₈ is C.

2. The peptide dimer according to 1, wherein each of said monomeric peptides comprises a sequence of amino acids

YX₂X₃X₄X₅GPX₆TWX₇X₈ (SEQ ID NO:253, in U.S. Pat.
No. 5,986,047)

where each amino add is indicated by standard one letter abbreviation; each of X₂ and X₆ is independently selected from any one of the 20 genetically coded L-amino acids; X₃ is C; X₄ is R, H, L, or W; X₅ is M, F, or I; X₇ is D, E, I, L, or V; and X₈ is C.

3. The peptide dimer according to 2, wherein each of said monomeric peptides comprises a sequence of amino acids

X₁YX₂X₃X₄X₅GPX₆TWX₇X₈X₉X₁₀X₁₁ (SEQ ID NO:254, in
U.S. Pat. No. 5,986,047)

where each amino acid is indicated by standard one letter abbreviation; each of X₁, X₂, X₆, X₉, X₁₀, and X₁₁ is independently selected from any one of the 20 genetically coded L-amino acids; X₃ is C; X₄ is R, H, L, or W; X₅ is M, F, or I; X₇ is D, E, I, L, or V, and X₈ is C.

4. The peptide dimer according to 3 wherein X₄ is R or H; X₅ is F or M; X₆ is I, L, T, M, orV; X₇ is D or V; X₉ is G K, L, Q, R, S, or T; and X₁₀ is A, G, P, R, or Y.

5. The peptide dimer according to 4, wherein X₁ is D, E, L, N, S, T, or V; X₂ is A, H, K, L, M, S, or T; X₄ is R or H; X₉ is K, R, S, or T; and X₁₀ is P.

6. The peptide dimer according to 5, wherein each of said monomeric peptides is GGTYSCHFGPLTWVCKPQGG (SEQ ID NO:8, in U.S. Pat. No.5,986,047).

7. The peptide dimer according to any one of 1-6, wherein the C-terminus of one of said monomeric peptides is covalently bound to the N-terminus of the other monomeric peptide.

8. The peptide dimer according to any one of 1-6, wherein the N-terminus of one of said monomeric peptides is covalently bound to the N-terminus of the other monomeric peptide.

9. The peptide dimer according ot any one of 1-6, wherein the cysteine residue of one of said monomeric peptides is covalently bound to the cysteine residue of the other monomeric peptide.

Suitable erythropoietin mimetic peptides also include those disclosed in U.S. Pat. No. 5,773,569 (which is incorporated herein by reference in its entirety), i.e., peptides of 10 to 40 or more amino acid residues in length and having the sequence X₃X₄X₅GPX₆TWX₇X₈ (SEQ ID NO:252, in U.S. Pat. No. 5,773,569) where each amino acid is indicated by standard one letter abbreviation; X₃ is C; X₄ is R, H, L, or W; X₅ is M, F, or I; X₆ is independently selected from any one of the 20 genetically coded L-amino acids; X₇ is D, E, I, L, or V; and X8 is C, which bind and activate the erythropoietin receptor (EPO-R) or otherwise act as an EPO agonist.

More specifically, the EPO mimetic peptide includes peptides having a peptide that satisfies the following requirements as a component:

1. A peptide of 10 to 40 amino acid residues in length that binds to erythropoietin receptor and comprises a sequence of amino acids X₃X₄X₅GPX₆TWX₇X₈ (SEQ ID NO:252, in U.S. Pat. No. 5,773,569) where each amino acid is indicated by standard one letter abbreviation; X₆ is independently selected from any one of the 20 genetically coded L-amino acids; X₃ is C; X₄ is R, H, L, or W; X₅ is M, F, or I; X₇ is D, E, I, L, or V; and X₈ is C.

2. The peptide according to 1 that comprises a sequence of amino acids YX₂X₃X₄X₅GPX₆TWX₇X₈ (SEQ ID:253, in U.S. Pat. No. 5,773,569) where each amino acid is indicated by standard one letter abbreviation; each of X₂ and X₆ is independently selected from any one of the 20 genetically coded L-amino acids; X₃ is C; X₄ is R, H, L, or W; X₅ is M, F, or I; X₇ is D, E, I, L, or V; and X₈ is C.

3. The peptide according to 2 that comprises a sequence of amino acids X₁YX₂X₃X₄X₅GPX₆TWX₇X₈X₉X₁₀X₁₁ (SEQ ID:254, in U.S. Pat. No. 5,773,569) where each amino acid is indicated by standard one letter abbreviation; each of X₁, X₂, X₆, X₉, X₁₀, and X₁₁ is independently selected from any one of the 20 genetically coded L-amino acids; X₃ is C; X₄ is R, H, L, or W; X₅ is M, F, or I; X₇ is D, E, I, L, or V, and X₈ is C.

4. The peptide according to 3 wherein X₄ is R or H; X₅ is F or M; X₆ is I, L, T, M, or V; X₇ is D or V; X₉ is G, K, L, Q, R, S, or T; and X₁₀ is A, G, P, R, or Y.

5. The peptide according to 4 wherein X₁ is D, E, L, N, S, T, or V; X₂ is A, H, K, L, M, S, or T; X₄ is R or H; X₉ is K, R, S, or T; and X₁₀ is P.

6. The peptide according to 1 that is selected from the group consisting of:

GGLYLCRFGPVTWDCGYKGG (SEQ ID NO:7, in U.S. Pat.
No. 5,773,569);
GGTYSCHFGPLTWVCKPQGG (SEQ ID NO:8, in U.S. Pat.
No. 5,773,569);
GGDYHCRMGPLTWVCKPLGG (SEQ ID NO:9, in U.S. Pat.
No. 5,773,569);
VGNYMCHFGPITWVCRPGGG (SEQ ID NO:10, in U.S. Pat.
No. 5,773,569);
GGVYACRMGPITWVCSPLGG (SEQ ID NO:11, in U.S. Pat.
No. 5,773,569);
VGNYMAHMGPITWVCRPGG (SEQ ID NO:12, in U.S. Pat.
No. 5,773,569);
GGPHHVYACRMGPLTWIC (SEQ ID NO:61, in U.S. Pat. No.
5,773,569);
GGTYSCHFGPLTWVCKPQ (SEQ ID NO:13, in U.S. Pat. No.
5,773,569);
GGLYACHMGPMTWVCQPLRG (SEQ ID NO:14, in U.S. Pat.
No. 5,773,569);
TIAQYICYMGPETWECRPSPKA (SEQ ID NO:15, in U.S. Pat.
No. 5,773,569);
YSCHFGPLTWVCK (SEQ ID NO:16, in U.S. Pat. No.
5,773,569);
and
YCHFGPLTWVC (SEQ ID NO:17, in U.S. Pat. No.
5,773,569).

7. The peptide according to 1 wherein the peptide is

GGTYSCHGPLTWVCKPQGG (SEQ ID NO:8, in U.S. Pat. No.
5,773,569).

The EPO mimetic peptide Hematide™ developed by Affymax, Inc., is also suitable. The EPO mimetic peptide EMP1 (GGTYSCHFGPLTWVCKPQGG-NH2, disulfide bond between (C6-C 15)) is also suitable.

The term “EPO production promoter” refers to a medicament that induces the production of a biological endogenous EPO by being administered in vivo, and examples thereof include a hypoxia-inducible factor (HIF) stabilizer and the like. It may have any structure as long as it has an HIF-stabilizing action. Specific examples thereof include FG2216 (named YM311 in Japan) developed by FibroGen Co., and the like. Further, it may be an EPO production-promoting agent having the mechanism of action other than that of a hypoxia-inducible factor proline hydroxylase inhibitor.

As a method of applying the active ingredient of the present invention to a pharmaceutical, oral administration or parenteral administration can be employed. However, upon administration, the active ingredient is mixed with a solid or liquid nontoxic carrier for pharmaceutical use which is suitable for the administration route such as oral administration or injection, whereby it can be administered in a common dosage form for a pharmaceutical preparation. Examples of such a pharmaceutical preparation include solid preparations such as tablets, granules, powders and capsules, liquid preparations such as solutions, suspensions and emulsions, lyophilized preparations, and the like. These pharmaceutical preparations can be prepared in a customary manner. Examples of the nontoxic carrier for pharmaceutical use include glucose, lactose, sucrose, starches, mannitol, dextrins, glycerides of fatty acids, polyethylene glycol, hydroxyethyl starches, ethylene glycol, polyoxyethylene sorbitan fatty acid esters, amino acids, gelatin, albumin, water, physiological saline, and the like. Further, a commonly used additive such as a stabilizer, a lubricant, an emulsifying agent, a binder, or a tonicity adjusting agent can be used as needed.

According to the present invention, a myeloerythroid progenitor differentiation inducer which has high safety, can be orally administered, and is widely used is provided.

It is expected that the bone marrow erythroid progenitor cell(s) differentiation inducer of the present invention enables prevention or treatment of anemia for patients with anemia, and clinical symptoms such as less incentive to work, fatigability, shortness of breath, lightheadedness and palpitation, each of which accompanies anemia, are improved. Further, if it can be orally administered, the pain due to subcutaneous or intravenous injection is not caused, and moreover, it can be easily taken daily at home without going to the hospital. Therefore, it is expected that the inducer can achieve reliable improvement of anemia. Further, arginine is an amino acid present in vivo, therefore it is expected that side effects accompanying its intake do not occur and treatment can be performed without concerning the worsening of the renal function.

For example, as for patients who have already been treated with rHuEPO, it is expected that by adding the active ingredient of the present invention thereto and administering the resulting agent to the patients for whom the required dose of EPO is less than the maximum dosage regimen, preferably 9000 IU/week, 450 IU/kg/week (e.g., in the U.S.), or 300 IU/kg/week (e.g., in Europe), the required dose of EPO is reduced, and the burden of the patients such as cost and pain due to subcutaneous or intravenous injection is reduced. Further, improvement of anemia is expected for EPO-unresponsive patients for whom the required dose of EPO is the maximum dosage regimen, preferably 9000 IU/week, or more. Further, also in the case where a treatment using the EPO mimetic peptide or EPO production promoter is widely performed in the future, it is expected that by adding the active ingredient of the present invention thereto and administering the resulting agent to patients, the required dose is reduced, etc.

Other features of the invention will become apparent in the course of the following descriptions of exemplary embodiments which are given for illustration of the invention and are not intended to be limiting thereof.

### EXAMPLES

### Example 1

### CFU-E-Colony Stimulating Activity of Arginine (EPO).

An in vitro mouse CFU-E colony assay was carried out in accordance with the following method. After a female BDF-1 mouse at 10 weeks of age (Charles River Japan, Inc.) was killed by the cervical dislocation method, bone marrow cells were isolated from the femur and suspended in an IMDM medium (Invitrogen) containing 10% FCS (JRH Bioscience Inc). The bone marrow cells were centrifuged at 1500 rpm for 10 minutes at 4° C. and the precipitated bone marrow cells were resuspended in an amino acid-free IMDM medium and the number of cells was measured. To a dish with a diameter of 3.5 cm (Nalgen Nunc, Inc. International), 1 ml of a methyl cellulose semi-solid medium in which the bone marrow cells were suspended { 1 IU/ml rHuEPO (Chugai Pharmaceutical Co., Ltd.), 100 μM 2-mercaptoethanol (Wako Pure Chemical Industries, Ltd.), 24% FCS (JRH Bioscience Inc.), 0.8% methyl cellulose (methyl cellulose containing IMDM solution M3134, Stem Cell Technologies, Inc.), 2% BSA (SIGMA-ALDRICH Japan K.K.), 2.2×10⁵ cells/ml of bone marrow cells} containing arginine at a concentration of 170 μM was added (which is equal to an IMDM medium whose concentration of the amino acid composition is one-third and which includes FCS at 24%). In addition to the above composition, a medium was prepared by additionally adding arginine so as to give a final concentration of 600 μM, and culturing was carried out under the conditions of 37° C. and 5% CO₂ for 48 hours. Then, the number of CFU-E colonies was measured using an inverted microscope. N=4 for each condition. Unpaired t-test was used for a statistical analysis.

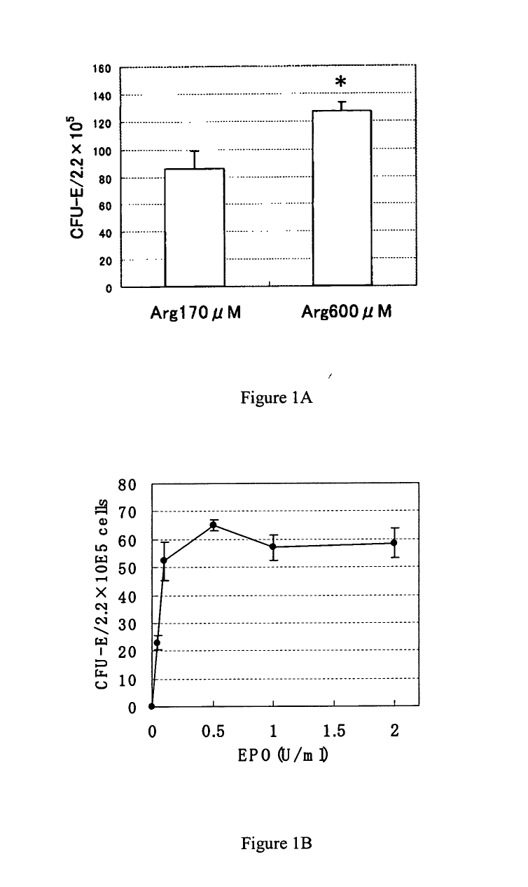

The results of CFU-E colony assay are shown in FIG. 1A. In the graph, * indicates P<0.05. The data is expressed as a mean value±SEM. The vertical axis indicates the number of CFU-E colonies per 2.2×10⁵ bone marrow cells.

In FIG. 1A, it is shown that the number of CFU-E increased when arginine was added to give a final concentration of 600 μM. From this, it was revealed that arginine directly acts on bone marrow cells and increases the number of CFU-E.

Because arginine increased the number of CFU-E under the condition in which the amount of EPO is sufficient, an action of enhancing the EPO effect and an action of reducing the dose of EPO can be expected. As shown in the results of FIG. 1B, which was obtained by carrying out an assay in the same condition as in FIG. 1A, because the amount for the highest action is 0.5 U/ml or higher, it is apparent that 1 U/ml of EPO is a sufficient amount. The same applies to a combination with an EPO mimetic peptide or an EPO production promoter. The details are mentioned below.

EPO is considered to act on erythropoiesis by binding to an EPO receptor present on the cell membrane of an erythroid stem cell. As shown in FIG. 1B, EPO increases erythroid stem cells CFU-E in a concentration-dependent manner and when it reaches a certain concentration, the maximum activity is reached, and even if the concentration is increased higher than that, the maximum activity remains unchanged. Although a sufficient amount of EPO exists, it was found in the present invention that arginine further increases CFU-E if it is allowed to act on concomitantly with EPO. From this, it is found that the site of action of EPO is different from that of arginine and that they exert a synergistic effect.

As mentioned above, EPO unresponsiveness means a state in which a sufficient effect is not expressed even if EPO is administered in an amount at which an effect is expected. However, even in the case like this where the effect of EPO alone is not sufficient, a high effect that exceeds the maximum activity of EPO can be obtained by administering arginine. Further, also for a patient other than an EPO-unresponsive patient, in the case where the required dose of EPO is high, the effect of EPO is enhanced by the concomitant use of arginine and, as a result, the dose of EPO can be reduced. In other words, arginine can be used as an “agent for enhancing the effect of EPO” or an “agent for reducing the dose of EPO”.

Because the site of action of EPO and an EPO mimetic peptide is a common EPO receptor, the relationship between EPO and arginine described above is exactly the same also in the case of the EPO mimetic peptide. In other words, arginine can be used as an “agent for enhancing the effect of an EPO mimetic peptide” or an “agent for reducing the dose of an EPO mimetic peptide.”

In a living body, EPO is produced in the kidney. When EPO required for erythropoiesis becomes insufficient accompanying a disease such as renal anemia, EPO is administered as a pharmaceutical product. However, even in renal anemia, it is not that the kidney could not produce EPO, but if the production of EPO is increased by using a renal EPO production promoter, the same therapeutic effect as that of EPO administration can be expected. Because the effect of arginine of the present invention is equal whether EPO is administered as a pharmaceutical product or it is endogenous EPO which is promoted to be produced in the kidney, a concomitant use with an EPO production promoter such as FG2216 mentioned above is also possible. In such a case, the effect shown in FIGS. 1A and 1B is expected and an effect can be enhanced more than that of administration of EPO alone or the dose of EPO can be reduced. In other words, arginine can be used as an “agent for enhancing an EPO production-promoting effect” or as an “agent for reducing the dose of an EPO production promoter.”

### Example 2

### CFU-E-Colony Stimulating Activity of Arginine (EPO Mimetic Peptide).

As a representative example of an EPO mimetic peptide, EMP 1 (GGTYSCHFGPLTWVCKPQGG-NH2, Disulfide bond between (C6-C 15)) described in Science, vol. 273, pp. 458-463 (1996), Table 1, etc. (which is incorporated herein by reference in its entirety) was prepared using the method described in Biochemistry, vol. 37(11), pp.3699-3710, at pp. 3700-3701 (which is incorporated herein by reference in its entirety).

After a female BDF-1 mouse at 10 weeks of age (Charles River Japan, Inc.) was killed by the cervical dislocation method, bone marrow cells were isolated from the femur and suspended in an IMDM medium (Invitrogen) containing 10% FCS (JRH Bioscience Inc). The bone marrow cells were centrifuged at 1500 rpm for 10 minutes at 4° C. and the precipitated bone marrow cells were resuspended in an amino acid-free IMDM medium and the number of cells was measured. To a dish with a diameter of 3.5 cm (Nalgen Nunc, Inc. International), 1 ml of a methyl cellulose semi-solid medium in which the bone marrow cells were suspended {30 μM EMP1, 100 μM 2-mercaptoethanol (Wako Pure Chemical Industries, Ltd.), 24% FCS (JRH Bioscience Inc.), 0.8% methyl cellulose (methyl cellulose containing IMDM solution M3134, Stem Cell Technologies, Inc.), 2% BSA (SIGMA-ALDRICH Japan K.K.), 1×10⁵ cells/ml of bone marrow cells} containing arginine at a concentration of 170 μM was added (which is equal to an IMDM medium whose concentration of the amino acid composition is one-third and which includes FCS at 24%). In addition to the above composition, a medium was prepared by additionally adding arginine so as to give a final concentration of 770 μM, and the culture was carried out under the conditions of 37° C. and 5% CO₂ for 48 hours. Then, the number of CFU-E colonies was measured using an inverted microscope. N=4 for each condition. Unpaired t-test was used for a statistical analysis.

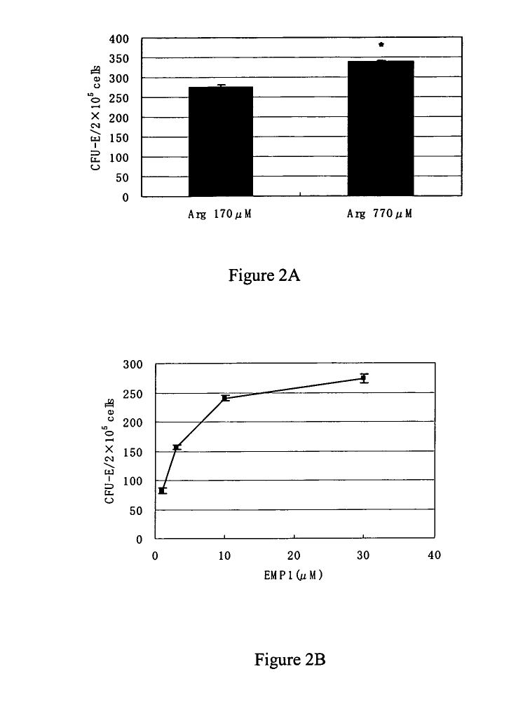

The results of CFU-E colony assay are shown in FIG. 2A. In the graph, * indicates P<0.05. The data is expressed as a mean value ±SEM. The vertical axis indicates the number of CFU-E colonies per 2×10⁵ bone marrow cells.

Because arginine increased the number of CFU-E under the condition in which the amount of EMP1 (EPO mimetic peptide) is sufficient, an action of enhancing EMP1 effect and an action of reducing the dose of EMP1 can be expected. As shown in the results of FIG. 2B, which was obtained by carrying out an assay in the same condition as in FIG. 2A, because the amount for the highest action is 10 μM or higher, it is apparent that 30 μM of EMP1 is a sufficient amount.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by reference, the same as if set forth at length.

1. A method for inducing bone marrow erythroid progenitor cells differentiation, comprising administering an effective amount of arginine to a subject in need thereof.

2. A method of treating and/or preventing anemia, comprising administering an effective amount of arginine to a subject in need thereof.

3. The method according to claim 2, wherein said arginine is administered in an amount of 0.1 to 12 g per day.

4. The method according to claim 2, wherein said arginine is administered in an amount of 0.5 to 6 g per day.

5. The method according to claim 2, wherein said subject undergoes hemodialysis or peritoneal dialysis.

6. The method according to claim 2, wherein said arginine is administered orally.

7. The method according to claim 2, wherein said arginine is administered enterally.

8. The method according to claim 2, wherein said subject suffers from renal anemia.

9. The method according to claim 2, wherein said subject is erythropoietin-unresponsive.

10. The method according to claim 9, wherein said subject exhibits an increase in Hct value of less than 3% after 4 to 8 weeks of administration of 9000 IU/week of EPO.

11. The method according to claim 9, wherein said subject does not exhibit an Hb level of 10 to 12 g/dl after 4 to 6 months of administration of 300 to 450 IU/kg/week of EPO.

12. The method according to claim 9, wherein said subject does not exhibit an Hb level increase of 2 g/dl and does not exhibit an Hb level of 10 to 12 g/dl after 2 months of administration of 150 to 300 IU/kg/week of EPO.

13. The method according to claim 2, further comprising administering an erythropoietin-like substance.

14. The method according to claim 13, wherein said erythropoietin-like substance is administered in an amount of 1500 to 9000 IU/week.

15. The method according to claim 13, wherein the erythropoietin-like substance is erythropoietin.

16. The method according to claim 13, wherein the erythropoietin-like substance is an erythropoietin mimetic peptide.

17. The method according to claim 13, wherein the erythropoietin-like substance is an erythropoietin production promoter.

18. The method according to claim 13, wherein said arginine and said erythropoietin-like substance are administered separately.

19. The method according to claim 13, wherein said arginine and said erythropoietin-like substance are administered in a combined composition.

20. The method according to claim 19, wherein the erythropoietin-like substance is erythropoietin.

21. The method according to claim 19, wherein the erythropoietin-like substance is an erythropoietin mimetic peptide.

22. The method according to claim 19, wherein the erythropoietin-like substance is an erythropoietin production promoter.

23. A method for enhancing the effect of an erythropoietin-like substance in treating and/or preventing anemia, comprising administering an effective amount of arginine to a subject in need thereof.

24. The method according to claim 23, wherein said arginine is administered in an amount of 0.1 to 12 g per day.

25. The method according to claim 23, wherein said arginine is administered in an amount of 0.5 to 6 g per day.

26. The method according to claim 23, wherein the erythropoietin-like substance is erythropoietin.

27. The method according to claim 23, wherein the erythropoietin-like substance is an erythropoietin mimetic peptide.

28. The method according to claim 23, wherein the erythropoietin-like substance is an erythropoietin production promoter.

29. A method for reducing the dose of an erythropoietin-like substance effective for treating and/or preventing anemia, comprising administering an effective amount of arginine to a subject in need thereof.

30. The method according to claim 29, wherein said arginine is administered in an amount of 0.1 to 12 g per day.

31. The method according to claim 29, wherein said arginine is administered in an amount of 0.5 to 6 g per day.

32. The method according to claim 29, wherein the erythropoietin-like substance is erythropoietin.

33. The method according to claim 29, wherein the erythropoietin-like substance is an erythropoietin mimetic peptide.

34. The method according to claim 29, wherein the erythropoietin-like substance is an erythropoietin production promoter.

35. A method of promoting hematopoiesis for an individual who stores his/her own blood, comprising administering an effective amount of arginine to said individual.

36. The method according to claim 35, wherein said arginine is administered in an amount of 0.1 to 12 g per day.

37. The method according to claim 35, wherein said arginine is administered in an amount of 0.5 to 6 g per day.


SEQUENCE LISTING (44 sequences)

SEQ ID NO: 1 - protein, 10 aa, Artificial Sequence
  Synthetic Peptide
  MISC_FEATURE (1)..(1): Xaa = C
  MISC_FEATURE (2)..(2): Xaa = R, H, L, or W
  MISC_FEATURE (3)..(3): Xaa = M, F, or I
  MISC_FEATURE (6)..(6): Xaa = any amino acid
  MISC_FEATURE (9)..(9): Xaa = D, E, I, L, or V
  MISC_FEATURE (10)..(10): Xaa = C
XXXGPXTWXX

SEQ ID NO: 2 - protein, 12 aa, Artificial Sequence
  Synthetic Peptide
  MISC_FEATURE (2)..(2): Xaa = any amino acid
  MISC_FEATURE (3)..(3): Xaa = C
  MISC_FEATURE (4)..(4): Xaa = R, H, L, or W
  MISC_FEATURE (5)..(5): Xaa = M, F, or I
  MISC_FEATURE (8)..(8): Xaa = any amino acid
  MISC_FEATURE (11)..(11): Xaa = D, E, I, L, or V
  MISC_FEATURE (12)..(12): Xaa = C
YXXXXGPXTWXX

SEQ ID NO: 3 - protein, 16 aa, Artificial Sequence
  Synthetic Peptide
  MISC_FEATURE (1)..(1): Xaa = any amino acid
  MISC_FEATURE (3)..(3): Xaa = any amino acid
  MISC_FEATURE (4)..(4): Xaa = C
  MISC_FEATURE (5)..(5): Xaa = R, H, L, or W
  MISC_FEATURE (6)..(6): Xaa = M, F, or I
  MISC_FEATURE (9)..(9): Xaa = any amino acid
  MISC_FEATURE (12)..(12): Xaa = D, E, I, L, or V
  MISC_FEATURE (13)..(13): Xaa = C
  MISC_FEATURE (14)..(16): Xaa = any amino acid
XYXXXXGPXTWXXXXX

SEQ ID NO: 4 - protein, 16 aa, Artificial Sequence
  Synthetic Peptide
  MISC_FEATURE (1)..(1): Xaa = any amino acid
  MISC_FEATURE (3)..(3): Xaa = any amino acid
  MISC_FEATURE (4)..(4): Xaa = C
  MISC_FEATURE (5)..(5): Xaa = R or H
  MISC_FEATURE (6)..(6): Xaa = F or M
  MISC_FEATURE (9)..(9): Xaa = I, L, T, M, E, or V
  MISC_FEATURE (12)..(12): Xaa = D or V
  MISC_FEATURE (13)..(13): Xaa = C
  MISC_FEATURE (14)..(14): Xaa = G, K, L, Q, R, S, or T
  MISC_FEATURE (15)..(15): Xaa = A, G, P, R, or Y
  MISC_FEATURE (16)..(16): Xaa = any amino acid
XYXXXXGPXTWXXXXX

SEQ ID NO: 5 - protein, 16 aa, Artificial Sequence
  Synthetic Peptide
  MISC_FEATURE (1)..(1): Xaa = D, E, L, N, S, T, or  V
  MISC_FEATURE (3)..(3): Xaa = A, H, K, L, M, S, or T
  MISC_FEATURE (4)..(4): Xaa = C
  MISC_FEATURE (5)..(5): Xaa = R or H
  MISC_FEATURE (6)..(6): Xaa = F or M
  MISC_FEATURE (9)..(9): Xaa = I, L, T, M, E, or V
  MISC_FEATURE (12)..(12): Xaa = D or V
  MISC_FEATURE (13)..(13): Xaa = C
  MISC_FEATURE (14)..(14): Xaa = K, R, S, or T
  MISC_FEATURE (15)..(15): Xaa = P
  MISC_FEATURE (16)..(16): Xaa = any amino acid
XYXXXXGPXTWXXXXX

SEQ ID NO: 6 - protein, 20 aa, Artificial Sequence
  Synthetic Peptide
GGLYLCRFGPVTWDCGYKGG

SEQ ID NO: 7 - protein, 20 aa, Artificial Sequence
  Synthetic Peptide
GGTYSCHFGPLTWVCKPQGG

SEQ ID NO: 8 - protein, 18 aa, Artificial Sequence
  Synthetic Peptide
GGTYSCHFGPLTWVCKPQ

SEQ ID NO: 9 - protein, 20 aa, Artificial Sequence
  Synthetic Peptide
GGDYHCRMGPLTWVCKPLGG

SEQ ID NO: 10 - protein, 20 aa, Artificial Sequence
  Synthetic Peptide
VGNYMCHFGPITWVCRPGGG

SEQ ID NO: 11 - protein, 20 aa, Artificial Sequence
  Synthetic Peptide
GGNYMCHFGPITWVCRPGGG

SEQ ID NO: 12 - protein, 20 aa, Artificial Sequence
  Synthetic Peptide
GGVYACRMGPITWVCSPLGG

SEQ ID NO: 13 - protein, 19 aa, Artificial Sequence
  Synthetic Peptide
VGNYMAHMGPITWVCRPGG

SEQ ID NO: 14 - protein, 18 aa, Artificial Sequence
  Synthetic Peptide
GGPHHVYACRMGPLTWIC

SEQ ID NO: 15 - protein, 18 aa, Artificial Sequence
  Synthetic Peptide
GGTYSCHFGPLTWVCKPQ

SEQ ID NO: 16 - protein, 20 aa, Artificial Sequence
  Synthetic Peptide
GGLYACHMGPMTWVCQPLRG

SEQ ID NO: 17 - protein, 22 aa, Artificial Sequence
  Synthetic Peptide
TIAQYICYMGPETWECRPSPKA

SEQ ID NO: 18 - protein, 13 aa, Artificial Sequence
  Synthetic Peptide
YSCHFGPLTWVCK

SEQ ID NO: 19 - protein, 11 aa, Artificial Sequence
  Synthetic Peptide
YCHFGPLTWVC

SEQ ID NO: 20 - protein, 20 aa, Artificial Sequence
  Synthetic Peptide
GGTYSCHFGPLTWVCKPQGG

SEQ ID NO: 21 - protein, 14 aa, Artificial Sequence
  Synthetic Peptide
GGCRIGPITWVCGG

SEQ ID NO: 22 - protein, 22 aa, Artificial Sequence
  Synthetic Peptide
LGRKYSCHFGPLTWVCQPAKKD

SEQ ID NO: 23 - protein, 20 aa, Artificial Sequence
  Synthetic Peptide
GGTASCHFGPLTWVCKPQGG

SEQ ID NO: 24 - protein, 20 aa, Artificial Sequence
  Synthetic Peptide
GGNYYCRFGPITFECHPTGG

SEQ ID NO: 25 - protein, 20 aa, Artificial Sequence
  Synthetic Peptide
GGEYLCRMGPMTWVCTPVGG

SEQ ID NO: 26 - protein, 20 aa, Artificial Sequence
  Synthetic Peptide
GGLYTCRMGPITWVCLPAGG

SEQ ID NO: 27 - protein, 20 aa, Artificial Sequence
  Synthetic Peptide
GGTTSCHFGPLTWVCKPQGG

SEQ ID NO: 28 - protein, 20 aa, Artificial Sequence
  Synthetic Peptide
GGTFSCHFGPLTWVCKPQGG

SEQ ID NO: 29 - protein, 20 aa, Artificial Sequence
  Synthetic Peptide
GGTYSCHFGALTWVCKPQGG

SEQ ID NO: 30 - protein, 20 aa, Artificial Sequence
  Synthetic Peptide
GGTYSCHFGPLAWVCKPQGG

SEQ ID NO: 31 - protein, 20 aa, Artificial Sequence
  Synthetic Peptide
GGTYSCHFAPLTWVCKPQGG

SEQ ID NO: 32 - protein, 20 aa, Artificial Sequence
  Synthetic Peptide
GGTYSCHFGPATWVCKPQGG

SEQ ID NO: 33 - protein, 20 aa, Artificial Sequence
  Synthetic Peptide
GGTYSCHFGPLTAVCKPQGG

SEQ ID NO: 34 - protein, 20 aa, Artificial Sequence
  Synthetic Peptide
GGTYSCHFGPLTFVCKPQGG

SEQ ID NO: 35 - protein, 16 aa, Artificial Sequence
  Synthetic Peptide
TYSCHFGPLTWVCKPQ

SEQ ID NO: 36 - protein, 14 aa, Artificial Sequence
  Synthetic Peptide
YSCHFGPLTWVCKP

SEQ ID NO: 37 - protein, 12 aa, Artificial Sequence
  Synthetic Peptide
SCHFGPLTWVCK

SEQ ID NO: 38 - protein, 19 aa, Artificial Sequence
  Synthetic Peptide
GGTYSCFGPLTWVCKPQGG

SEQ ID NO: 39 - protein, 18 aa, Artificial Sequence
  Synthetic Peptide
TYSCHFGPLTWVCKPQGG

SEQ ID NO: 40 - protein, 12 aa, Artificial Sequence
  Synthetic Peptide
YSCHFGPLTWVC

SEQ ID NO: 41 - protein, 20 aa, Artificial Sequence
  Synthetic Peptide
GGTYSCHFGPLTFVCKPQGG

SEQ ID NO: 42 - protein, 8 aa, Artificial Sequence
  Synthetic Peptide
HFGPLTWV

SEQ ID NO: 43 - protein, 13 aa, Artificial Sequence
  Synthetic Peptide
YSCHFGALTWVCK

SEQ ID NO: 44 - protein, 20 aa, Artificial Sequence
  Synthetic Peptide
GGTYSEHFGPLTWVKKPQGG